(19) United States
(12) Patent Application Publication
(10) Pub. No.: US 2007/0292956 A1
(43) Pub. Date: Dec. 20, 2007

(54) Sensing Element for a Biosensor

(76) Inventor: Daniel Henry Densham, Exeter (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/835,124

(22) Filed: Aug. 7, 2007

Related U.S. Application Data
(63) Continuation of application No. 10/564,795, filed on Jun. 6, 2006.

(30) Foreign Application Priority Data
Jul. 15, 2003 (GB) 0316553.7

(51) Int. Cl. G01N 33/53; C12M 1/34

(52) U.S. Cl. 436/2; 422/50; 422/68.1; 435/287.2; 977/900

(57) ABSTRACT

A sensing element for use in a biosensor comprises a matrix of discrete particles formed from a material capable of supporting surface electromagnetic waves, the particles having a biologically active molecule bound thereto.

### FIELD OF THE INVENTION

The present invention relates to a system for detecting a physical, chemical or biochemical reaction, and in particular to a system in which surface electromagnetic waves (SEWs) interact with a specimen involved in the reaction.

### BACKGROUND OF THE INVENTION

Biosensors incorporating surface electromagnetic wave technology (and, in particular, surface plasmon resonance-SPR-sensors) are increasingly gaining popularity in pharmaceutical, medical and environmental applications as well as in biochemical research. These sensors require no labelling and offer the possibility of real-time monitoring of binding events. They are based on the sensitivity of surface electromagnetic waves (SEW) to the refractive index of the thin layer adjacent to the surface where the SEW propagates. In a typical biosensor application, one binding partner is immobilized on the surface (often called a target) and the other partner is flowed across the surface. As binding occurs, the accumulation or redistribution of mass on the surface changes the local refractive index that can be monitored in real time by the sensor.

Several methods of SPR registration have been proposed and realized in biosensors. The most popular methods are based on the Kretschmann-Raether configuration where intensity of the light reflected from the sensor is monitored. This technique, considered to be one of the most sensitive, is described in J. Homola et al, Sensors and Actuators B 54, p. 3-15 (1999) and has a detection limit of 5×10⁻⁷ refractive index units. Measuring SPR phase changes can further increase the sensitivity of the sensor by one or two orders of magnitude. This is described in Nelson et al, Sensors and Actuators B 35-36, p. 187 (1996) and in Kabashkin et al, Optics Communications 150, p. 5 (1998). Prior art interferometric devices such as a Mach Zehnder device have been configured to measure variations in the refractive index at the sensor surface via phase shifts. This is disclosed in WO01/20295. The configuration requires four independent components and is sensitive to sub-wavelength relative displacements of these components and hence very small mechanical and environmental perturbations. A mechanically more robust monolithic interferometric design is outlined in WO03014715.

For SEW techniques to work satisfactorily, it is important to use a sensing element (sensing surface) which provides sensitivity for the detection of changes in local refractive index. A typical sensing element comprises a glass substrate onto which a thin metal layer is formed. Although useful, there is still a general need for improvements to the design of biosensors for increasing the sensitivity of detection.

### SUMMARY OF THE INVENTION

The present invention is based on the realisation that providing a sensing element composed of discrete particles will improve the sensitivity of SEW techniques.

According to a first aspect of the invention, a sensing element for use in a biosensor comprises a matrix of discrete particles formed from a material capable of supporting surface electromagnetic waves, the particles having a biologically molecule bound thereto.

According to a second aspect of the invention, an apparatus for detecting a physical, chemical or biochemical reaction, comprises a coherent radiation source for producing an incident wave; a sensing element for supporting a molecule to be analysed, the element being as defined above; and a detector for monitoring changes in radiation reflected from the sensing element.

According to a third aspect of the invention, a sensing element or apparatus as defined above is used in an assay to detect changes in the molecule bound to the sensing element.

According to a fourth aspect of the invention, a method for monitoring a molecule undergoing a physical, chemical or biochemical reaction occurring on a sensing element, comprises the steps of: applying electromagnetic radiation to a sensing element having the molecule bound thereto; and monitoring changes in radiation reflected from the sensing element, wherein the sensing element is as defined above.

### DESCRIPTION OF THE DRAWINGS

The invention is described herein with reference to the accompanying figure, wherein:

FIG. 1 is a schematic illustration of a matrix formed by metal or semi conductor nanoparticles (II) interconnected by a linker molecule, and having a biological molecule (antibody) attached.

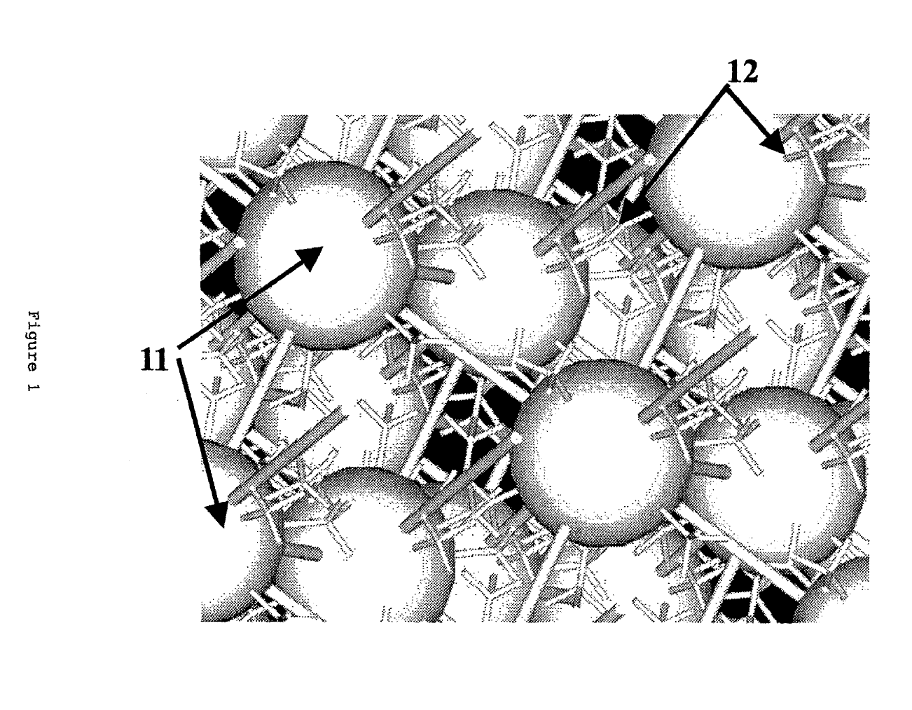

### DESCRIPTION OF THE INVENTION

The present invention is based on the realisation that the provision of a matrix of particles that support surface electromagnetic waves will enhance the energy/phase changes which occur as the molecule being studied undergoes a physical, chemical or biochemical reaction. The particles for use in the present invention are preferably comprised of a metal capable of supporting surface electromagnetic waves, e.g. surface plasmons. Although any metal may be used as part of the particles, it is preferable to use a metal that is physically inert, e.g. gold, copper, aluminium or silver. Preferably gold is used as the metal for the particles due to its highly inert characteristic. The matrix may comprise a combination of different metallic particles, e.g. gold and silver, although it is preferred to have a uniform matrix of a single metallic substance.

The size of the particles will depend largely on the sensing element required, depending on the SEW technique to be employed. The particles will typically be from 1 nanometre to several microns in diameter, more preferably the particles will be submicron in size, typically from 5 nm to 50 nm in diameter i.e. the particles will be nanoparticles.

The matrix comprises a plurality of the particles with the matrix mounted on the surface of a supporting transparent dialectric material. The sensing element will therefore comprise a first section of a first thickness comprised of the matrix overlaid on a second section of greater thickness, formed from the transparent dialectric material. The instant radiation beam is scattered on the matrix material and the scattering will vary depending on the reaction taking place with the molecule understudy.

The discrete particles may be interlinked by the use of a bridging chemical or polymer material. This will help retain a stable structure for the matrix. Suitable methods for linking the particles together will be known to those skilled in the art. For example, covalent linking agents including thiol reagents may be used to link gold particles. The linking molecules may be attached covalently or non-covalently and may be linked to the particle directly or through an intermediate linker molecule.

The matrix layer, when overlaid on the surface of the dialectric material, may have a thickness suitable for use in an SEW technique. The conventional thickness for use in SEW techniques is approximately 500 nm, preferably no more than 100 nm and more preferably less than 50 nm thickness. The matrix may therefore form a uniform layer on the surface of the dialectric material of only a single particle thickness or alternatively there may be multiple particles stacked within the matrix.

Any suitable biologically-active molecule may be immobilised to the particles. SEW techniques are used to study a wide range of molecules, including prokaryotic and eukaryotic cells, including pathogenic bacterial cells, polymer molecules, including proteins and chemical molecules including pharmaceutical drugs. The invention is of particular use when using proteins as the molecule. For example, the molecule bound to the particles may be an antibody or an antigen and the reaction being studied may be the interaction of the antibody or antigen with its respective binding partner or ligand. The molecule may also be a biological receptor, with the SEW technique used to detect the interaction of the receptor with its ligand.

In a particularly preferred embodiment, the molecule attached to the particle is an enzyme that interacts with and processes along a polynucleotide sequence. For example, the enzyme may be a polymerase enzyme and the SEW technique may be used to determine the sequence of a polynucleotide by measuring the conformational changes that occur on a polymerase enzyme as the polymerase incorporates specific nucleotides onto a target polynucleotide sequence. The use of surface plasmon resonance spectroscopy to determine the sequence of polynucleotides in this way is described in WO99/05315.

The polymerase used in the invention may be of any known type. For example, the polymerase may be any DNA-dependent DNA polymerase. If the target polynucleotide is a RNA molecule, then the polymerase may be a RNA-dependent DNA polymerase, i.e. a reverse transcriptase, or a RNA-dependent RNA polymerase, i.e. RNA replicase.

Alternatively, the enzyme may be a helicase, which may also be used to determine the sequence of a target polynucleotide, as described in WO00/60114.

The molecule may be bound directly or indirectly to a particle. It is recognised that there are often difficulties in attaching proteins directly to metallic surfaces, which often result in partial inactivation of the protein. Accordingly, the present invention envisages utilising intermediate linking molecules to bind proteins (or other molecules) to the particles. It is within the scope of the invention to coat the particles prior to the attachment of molecules with a substance that provides a suitable barrier between the molecule and the particle. For example, the particles may be coated with a hydrogel as disclosed in U.S. Pat. No. 5,436,161. The hydrogel may, for example, be a polysaccharide such as agarose, dextran, carrageenan, alginic acid, starch, cellulose, or derivatives thereof, or a water-swellable organic polymer such as, e.g. polyvinyl alcohol, polyacrylic acid, polyacrylimide, and polyethanolglycol. The hydrogel may be derivatised to contain hydroxyl, carboxyl, amino, aldehyde, carbonyl, epoxy or vinyl groups for binding the molecule. The sensing element may be used in any biosensor apparatus which can generate and detect surface electromagnetic waves. The preferred SEW technique is surface plasmon resonance (SPR). However, other techniques may also be used, for example, total internal reflectance fluorescence (TIRF), attenuated total reflection (ATR), frustrated total reflection (FTR), Brewster angle reflectometry, scattered total internal reflection (STIR) and evanescent wave ellipsometry.

Surface plasmon resonance is the preferred method and review of SPR techniques is provided in European Patent Publication No. 0648328.

The content of each of the publications disclosed herein is incorporated herein by reference.

We claim:

1. A sensing element, comprising a matrix of discrete particles formed from a material capable of supporting surface electromagnetic waves, the particles having a biologically active molecule bound thereto.

2. The sensing element according to claim 1, wherein the particles are metallic.

3. The sensing element according to claim 1, wherein the particles are gold.

4. The sensing element according to claim 1, wherein the particles are of sub-micron diameter.

5. The sensing element according to claim 4, wherein the diameter is from 5 nm to 50 nm.

6. The sensing element according to claim 1, wherein the particle matrix forms a surface on a dialectric substrate.

7. The sensing element according to claim 6, wherein the dialectric substrate is glass.

8. The sensing element according to claim 1, wherein the particles are linked via a polymer molecule.

9. The sensing element according to claim 1, wherein the biologically active molecule is a protein.

10. The sensing element according to claim 9, wherein the protein is a polymerase enzyme.

11. An apparatus for detecting a physical, chemical or biochemical reaction, comprising
a coherent radiation source for producing an incident wave;
a sensing element for supporting a molecule to be analyzed, the element comprising a matrix of discrete particles formed from a material capable of supporting surface electromagnetic waves, the particles having a biologically active molecule bound thereto; and
a detector for monitoring changes in radiation reflected from the sensing element.

12. Use of a sensing element of claim 1, or an apparatus comprising the sensing element and a detector for monitoring changes in radiation reflected from the sensing element, in an assay to detect changes in the molecule bound to the sensing element.

13. A method for monitoring a molecule undergoing a physical, chemical or biochemical reaction occurring on a sensing element, comprising:
applying electromagnetic radiation to a sensing element having one molecule bound thereto; and
monitoring changes in radiation reflected from the sensing element, wherein the sensing element comprises a matrix of discrete particles formed from a material capable of supporting surface electromagnetic waves, the particles having a biologically active molecule bound thereto.
